(19) United States
(12) Patent Application Publication
(10) Pub. No.: US 2026/0033525 A1
(43) Pub. Date: Feb. 5, 2026

(54) DRIED PROPIONATE FERMENT

(71) Applicant: PURAC Biochem B.V., Gorinchem (NL)

(72) Inventors: Jeff Bradley BOTTS, Lenexa, KS (US); Jacoba EELDERINK, Gorinchem (NL); Pauline Ludivine FOLCH, Gorinchem (NL); Nicolas Jean Yves GUILBOT, Gorinchem (NL); Johannes Wilhelmus Otto SALARI, Gorinchem (NL); Tithira Tirangika WIMALASENA, Gorinchem (NL); Mary Christine WINGER, Lenexa, KS (US)

(73) Assignee: PURAC Biochem B.V., Gorinchem (NL)

(21) Appl. No.: 19/356,602

(22) Filed: Oct. 13, 2025

Related U.S. Application Data
(63) Continuation of application No. PCT/EP2024/056586, filed on Mar. 12, 2024.

(30) Foreign Application Priority Data
Apr. 14, 2023 (EP) 23168034.9
Oct. 13, 2023 (EP) 23203618.6

(51) Int. Cl. A23L 33/16; A23L 33/135; C12P 3/00; C12P 7/62

(52) U.S. Cl. CPC A23L 33/16; A23L 33/135; C12P 3/00; C12P 7/62

(57) ABSTRACT

The present invention provides a dried fermentate that comprises, per kg of dry, matter: 4.7-8.1 mol of propionate; 1.2-3.0 mol of calcium cation; 2.4-7.8 mol of alkali metal cation selected from sodium, potassium and combinations thereof; and 1-50 grams glucose equivalent of hydrolysed glucan; calcium cation and propionate being present in the dried fermentate in a molar ratio of 0.2 to 0.6. Methods of producing the dried fermentate are also provided.

### CROSS-REFERENCE TO RELATED APPLICATIONS

The present application is a continuation of International Application No. PCT/EP2024/056586, filed Mar. 12, 2024, which claims priority to European Patent Application No. 23168034.9, filed Apr. 14, 2023, and European Patent Application No. 23203618.6, filed Oct. 13, 2023, all of which are hereby incorporated by reference herein in their entireties.

### TECHNICAL FIELD OF THE INVENTION

The present invention relates to a dried fermentate comprising calcium propionate.

The dried fermentate of the present invention has a very high propionate content and may suitably be applied as a preservative in e.g., food products and cosmetic products.

The invention further relates to a process comprising:
providing an aqueous fermentation medium comprising hydrolysed glucan;
inoculating the fermentation medium with propionic acid producing micro-organisms;
incubating the inoculated fermentation medium to produce a fermentate while adding an alkalising agent to maintain pH within a predefined range.

The invention also relates to the use of the dried fermentate in a food product or cosmetic product.

### BACKGROUND OF THE INVENTION

Preservatives extend the shelf life of baked goods by inhibiting the growth of rope bacteria and moulds. Rope bacteria (Bacillus pumilus) can be carried through the baking process and cause an unpleasant texture and odour inside yeast-raised products like bread. Moulds can contaminate products after baking and then grow on the outside surface. Mould contamination is considered a serious problem amongst bakers, and conditions commonly found in baking present near-optimal conditions for mould growth.

Acidity decreases the chance of spoilage. Most preservatives have acids as an active ingredient. Preservatives can be classified according to the kind of acid they contain: mineral acid, organic acid, or fatty acid. Mineral acids (like sulfuric, hydrochloric, and phosphoric) are the strongest acids and so have the greatest effect on pH. But they work only by lowering pH and do not contain any other inhibitors. Organic acids (like lactic, benzoic, and tartaric) have less effect on pH than do mineral acids but are better preservatives because they have an additional inhibitory activity. Fatty acids (like acetic, sorbic, and propionic) are a specific kind of organic acid with an even stronger inhibitory activity.

Calcium propionate is used as a preservative in a wide variety of products, including: bread, other baked goods, processed meat, whey, and other dairy products. Propionates prevent microbes from producing the energy they need, like benzoates do. However, unlike benzoates, propionates do not require an acidic environment.

Propionate is a commercially valuable carboxylic acid produced through microbial fermentation. Biological propionate production is limited by high downstream purification costs.

WO 2010/097362 describes a method for manufacturing a mixed solution comprising propionate and acetate, which comprises the steps of
providing a fermentation product comprising propionate, acetate, and carbonate-related compounds,
adding acid to the fermentation product to obtain an acidified fermentation product comprising propionate and acetate with a pH in the range of 2.5 to below 8.

WO 2016/146721 describes a process for manufacturing propionate products through fermentation, comprising the steps of:
fermenting a carbon source selected from sugars and lactate in a fermentation medium by means of a propionic acid producing micro-organism to provide a first fermentation broth comprising a propionate salt,
recovering propionic acid producing micro-organism from the first fermentation broth,
subjecting the first fermentation broth from which propionic acid producing micro-organism have been recovered to a water removal step to form a first propionate salt product,
fermenting a carbon source comprising glycerol with the propionic acid producing micro-organism recovered from the first fermentation broth in the presence of an inorganic alkaline salt to provide a second fermentation broth comprising a propionate salt,
subjecting the second fermentation broth to a purification step comprising at least one precipitation step, to form a second propionate salt product.

WO 2019/108064 describes an organic acid preservative system comprising an acetate component and a propionate component, wherein the acetate component and the propionate component make up at least 85 wt. % of the total amount of carboxylic acids and carboxylic acid salts in the preservative system and wherein the propionate component makes up 12 to 50 wt. % of said total amount of carboxylic acids and carboxylic acid salts.

CN 115 678 925 describes a method of preparing calcium propionate by fermenting a medium containing 0.1% Na₂HPO₄ and 0.1% MgSO₄.7H₂O with Propionibacterium, followed by membrane filtration, reaction of the isolated propionate with calcium hydroxide, evaporation and crystallisation.

CN 112 176 005 describes a method for producing calcium propionate comprising fermenting a fermentation medium with an acid-producing strain while maintaining pH between 7 and 2.5, sterilisation, nanofiltration and addition of an aqueous solution of calcium hydroxide. After the reaction is completed, the mixture is concentrated and dried to obtain solid calcium propionate.

### SUMMARY OF THE INVENTION

The inventors have discovered that a dried propionate fermentate with a very high propionate content and a bland taste can be obtained if a carefully balanced combination of alkaline calcium salt and alkaline alkalimetal salt is used in the preparation of the fermentate.

The dried fermentate of the present invention comprises per kg of dry matter:
4.7-8.1 mol of propionate;
1.2-3.0 mol of calcium cation;
2.4-7.8 mol, of alkali metal cation selected from sodium, potassium and combinations thereof;
1-50 grams glucose equivalent of hydrolysed glucan; calcium cation and propionate being present in the dried fermentate in a molar ratio of 0.2 to 0.6.

Besides the intrinsic buffer capacity of the acid salts present therein, the dried fermentate of the present invention has limited buffer capacity, probably due to the removal of buffering components that are contained in biomass, such as protein. This reduced buffer capacity is advantageous because fluctuations in buffer capacity observed between production batches are effectively minimised. Also, in case pH adjustment is required when the dried fermentate is applied in a food product, less acid or base is needed to achieve such pH adjustment.

The invention also provides a process for producing a fermentate, said process comprising the following steps:
providing an aqueous fermentation medium comprising 30-200 g/L glucose equivalent of hydrolysed glucan;
inoculating the fermentation medium with a propionic acid producing micro-organism;
incubating the inoculated fermentation medium for at least 20 hours to produce a fermentate while adding an alkalising agent to maintain pH within the range of 5.0 to 7.5, said alkalising agent including an alkaline water-soluble alkalimetal salt and an alkaline water-soluble calcium salt;

wherein the fermentate at the end of the incubation step comprises calcium cation and propionate in a molar ratio of 0.3 to 0.8 and alkalimetal cation and propionate in a molar ratio of 0.3-1.4.

The fermentate obtained by this process offers the advantage that it introduces less bitterness than existing calcium propionate fermentates when used to provide a certain propionate content in a final product. As a result, the present fermentate can suitably be applied in higher concentrations than existing propionate fermentates and thus achieve better preservation.

The invention also concerns a method of preserving a food product or cosmetic product, said method comprising addition of the dried fermentate of the present invention in a concentration of 0.1-5% by weight of the food product.

Yet another aspect of the invention concerns a food product or a cosmetic product that is obtained by aforementioned preservation method.

### DETAILED DESCRIPTION OF THE INVENTION

A first aspect of the invention concerns a dried fermentate having a moisture content of less than 15 wt. % and comprising per kg of dry matter:
4.7-8.1 mol of propionate;
1.2-3.0 mol of calcium cation;
2.4-7.8 mol, of alkali metal cation selected from sodium, potassium and combinations thereof;
1-50 grams glucose equivalent of hydrolysed glucan; calcium cation and propionate being present in the dried fermentate in a molar ratio of 0.2 to 0.6.

The term “propionate” as used herein encompasses propionic acid as well as its salts. Likewise, the terms “acetate”, “succinate” and “lactate” also encompass bot the acid forms and salt forms.

The term “glucan” as used herein refers to a linear or branched polymer consisting of glucose monomeric units. Examples of glucans include amylose, amylopectin, cellulose and glycogen.

Concentrations of hydrolysed glucan expressed as “glucose equivalent” refer to the total amount of glucose present in hydrolysed glucan, including both free glucose and glucose that is contained in hydrolysis fragments that have a degree of polymerisation of two or more. The amount of glucose equivalent present in a hydrolysed glucan can be determined by completely hydrolysing the hydrolysed glucan and measuring the glucose content.

The dried fermentate of the present invention preferably has a moisture content of less than 12 wt. %, more preferably a moisture content of 1-10 wt. %.

Propionate is preferably contained in the dried fermentate of the present invention in a concentration of 5.2-7.8 mol per kg of dry matter, most preferably in a concentration of 5.8-7.2 mol per kg of dry matter.

The dried fermentate preferably contains calcium cation in a concentration of 1.4-2.8 mol per kg of dry matter, most preferably in a concentration of 1.7-2.5 mol per kg of dry matter.

Hydrolysed glucan is preferably contained in the dried fermentate in a concentration of 2-30 grams glucose equivalent per kg of dry matter, most preferably 3-15 grams glucose equivalent per kg of dry matter.

According to a particularly preferred embodiment, the dried fermentate contains calcium cation and propionate in a molar ratio of 0.2 to 0.0.5, most preferably in a molar ratio of 0.22 to 0.4.

The hydrolysed glucan in the dried fermentate is preferably selected from hydrolysed amylose, hydrolysed amylopectin, hydrolysed cellulose, hydrolysed glycogen and combinations thereof. Most preferably the hydrolysed glucan comprises hydrolysed amylose and/or hydrolysed amylopectin. Both amylose and amylopectin are starch components.

The hydrolysed glucan preferably comprises at least one or more saccharides selected from glucose, maltose, isomaltose, maltotriose, maltotetraose and combinations thereof. According to a particularly preferred embodiment, the dried fermentate contains, per kg of dry matter, 0.5-10 grams of one or more saccharides selected from glucose, maltose, isomaltose, maltotriose, maltotetraose and combinations thereof, most preferably 0.8 to 5 grams of one or more saccharides selected from glucose, maltose, isomaltose, maltotriose, maltotetraose and combinations thereof.

According to a preferred embodiment, the dried fermentate of the present invention comprises DNA from a propionic acid producing micro-organism, preferably from a bacterium belonging to the genera Propionibacterium and Acidipropionibacterium, more preferably from a bacterium selected from Acidipropionibacterium acidipropionici, Propionibacterium freudenreichii, Propionibacterium shermanii, Acidipropionibacterium thoenii, Acidipropionibacterium jensenii and combinations thereof. The presence of DNA from a propionic acid producing micro-organism can suitably be determined using a PCR test.

In a particular preferred embodiment, the dried fermentate comprises per kg of dry matter 3.0-7.44 mol, more preferably 4.0-7.0 mol, most preferably 5.3-6.5 mol of alkali metal cation selected from sodium, potassium and combinations thereof.

In another preferred embodiment, the dried fermentate comprises per kg of dry matter at least 3.4 mol, more preferably at least 4.0 mol, most preferably at least 5.3 mol of sodium cation.

Besides propionate the dried fermentate of the present invention may comprise other organic acid salts, such as acetate and/or succinate.

Preferably, the dried fermentate contains, per kg of dry matter, 1.5-3.0 mol, more preferably 1.7-2.8 mol, most preferably 1.9-2.6 mol of acetate.

Succinate is preferably present in the dried fermentate in a concentration of 0.4-1.2 mol per kg of dry matter, more preferably of 0.5-1.1 mol per kg of dry matter, most preferably of 0.55-1.0 mol per of dry matter.

The combination of propionate, acetate and succinate is preferably contained in the dried fermentate in a concentration of 7.5-11.0 mol per kg of dry matter, more preferably of 8.2-10.5 mol per kg of dry matter, most preferably 8.7-10.2 mol per kg of dry matter.

The dried fermentate preferably contains carbonate in a concentration of 0-20 mmol per kg of dry matter, more preferably in a concentration 0-10 mmol per kg of dry matter, most preferably in a concentration of 0-5 mmol per kg of dry matter.

Further, in a preferred embodiment, the dried fermentate contains calcium cation and alkali metal cation in a molar ratio of 0.25 to 0.62, more preferably 0.30 to 0.55, most preferably 0.32 to 0.52.

In accordance with another preferred embodiment, the combination of propionate, acetate, calcium cation and alkali metal cation constitutes 90-99 wt. %, more preferably 92-98 wt. %, most preferably 93-97 wt. % of dry matter contained in the fermentate.

According to a particularly preferred embodiment, the dried fermentate contains propionate in a concentration X, acetate in a concentration Y, succinate in a concentration Z, calcium cation in a concentration A, sodium cation in a concentration B and potassium in a concentration C, wherein all said concentrations are expressed in mol/kg of dry matter and wherein 0.8≤ (X+Y+2Z)/(2A+B+C)≤1.2, preferably 0.85≤(X+Y+2Z)/(2A+B+C)≤1.15 and more preferably 0.90≤(X+Y+2Z)/(2A+B+C)≤1.12.

The dried fermentate of the present invention advantageously has a near neutral pH. Accordingly, in a preferred embodiment, dispersion of 100 grams of fermentate in 1 litre of water of 20° C. yields an aqueous liquid having a pH in the range of 6 to 8.

The dried fermentate of the present invention is largely water soluble. Preferably, when 100 grams of the fermentate is dispersed in 1 litre of water of 20° C. at least 95 wt. %, more preferably at 98 wt. % and most preferably at least 99 wt. % of the fermentate is dissolved.

The protein content of the dried fermentate preferably is in the range of 0-30 grams per kg of dry matter, more preferably in the range of 0-10 grams per kg of dry matter, most preferably in the range of 0-5 grams per kg of dry matter.

The dried fermentate preferably is a powder, wherein at least 80 wt. % of the particles have a diameter of no more than 500 μm. More preferably, the dried fermentate is a powder wherein at least 80 wt. % of the particles have a diameter in the range of 40 to 400 μm. The particle size distribution of the dried fermentate may suitably be determined by means of laser diffraction.

As mentioned above, the dried fermentate of the present invention offers the advantage that, besides the theoretical buffer capacity of the acid salts present in the fermentate, it has limited actual buffer capacity. The dried fermentate of the present invention preferably has a mean dynamic buffer capacity (β̅) of less than 27 mM/g, more preferably of 12-26 mM/g and most preferably of 16-25 mM/g. Static buffer capacity (β) is defined as the mmoles of HCl per gram of dried fermentate that is necessary to change the pH of a solution of said dried fermentate (0.2 g/100 ml of water) by 1. The buffer capacity (β) of a dried fermentate as a function of pH can be determined by titrating an aqueous fermentate solution (0.2 gram of dried fermentate in 100 ml of water) with a 0.1N HCl solution to reduce the pH to 2.5. The mean dynamic buffer capacity (β̅) which accounts for the impact of the volume of titrant can easily be determined by plotting the buffer capacity (β) as a function of pH and applying the mean volume theorem to the titration data according to the calculations below. The mean dynamic buffer capacity (β̅) better illustrates differences in fermentates that impact their functionality in products.

$$\begin{matrix}\overline{β} = \frac{1}{m} \cdot \frac{1}{{pH}_{1} - {pH}_{2}} \cdot {\int }_{p {H}_{1}}^{p {H}_{2}} β ( pH ) \cdot d pH \\ β ( pH ) = \vert \frac{d c}{d V} \cdot \frac{d V}{d pH} \vert\end{matrix}$$

where m=mass of the sample, c=concentration of sample and V=volume of titrant.

Dried fermentates that contain significant amounts of buffering components other than organic acid salts typically have a mean dynamic buffer capacity that is higher than the mean dynamic buffer capacity of the dried fermentates of the present invention. Also, the pH of the pKa of these dried fermentates is usually higher than the pH of the pKa of the dried fermentates of the present invention.

As mentioned herein before, the dried fermentate has a desirable taste profile. In particular, the dried fermentate has a low bitterness intensity. Preferably, a solution that is obtained when the dried fermentate is dissolved in demineralised water of 20° C. to achieve a propionate concentration of 140 mmol/L has a bitterness score of less than 50 on a line scale from 0 to 100. This bitterness score can be measured using conventional sensory methods such as difference testing with anchored line scales. For example, a ranking test with an instruction to rank the samples for bitterness intensity using a line scale anchored with “low” on one end and “high” on the other can be used to compare and evaluate pairs of samples. The responses can be captured electronically with software such as EyeQuestion® and analyzed with software such as EyeOpenR. The software assigns values from 0 for the end of the scale labelled “low” and 100 for the end of the scale labelled “high” and then determines the degree of statistical significance for the values by compared the scores of the panelists. For such difference and ranking tests, the use of trained panelists is not needed.

Furthermore, the taste profile of the dried fermentate contributes to desirable hedonic scores for food products that include the dried fermentate. In some embodiments, the food product comprising the dried fermentate in a concentration that provides 140 mmol propionate per kg has a hedonic liking score not significantly different from a food prod140 mmol/L uct comprising calcium propionate in the same molar concentration. For hedonic testing, a questionnaire with a nine-point liking scale can be used. The nine-points include: Dislike Extremely, Dislike Very Much, Dislike Moderately, Dislike Slightly, Neither Like Nor Dislike, Like Slightly, Like Moderately, Like Very Much, and Like Extremely. A range of attributes can be evaluate for liking includes overall liking, aroma liking, flavour liking, and after taste liking.

The panelist responses can be captured and analyzed electronically with software such as EyeQuestion® and EyeOpenR.

Another aspect of the invention relates to a process of preparing a fermentate, preferably a dried fermentate according to the present invention, said process comprising the following steps:
providing an aqueous fermentation medium comprising 30-200 g/L glucose equivalent of hydrolysed glucan;
inoculating the fermentation medium with a propionic acid producing micro-organism;
incubating the inoculated fermentation medium for at least 20 hours to produce a fermentate while adding an alkalising agent to maintain pH within the range of 5.0 to 7.5, said alkalising agent including an alkaline water-soluble alkalimetal salt and an alkaline water-soluble calcium salt;

wherein the fermentate at the end of the incubation step comprises calcium cation and propionate in a molar ratio of 0.2, to 0.8 and alkalimetal cation and propionate in a molar ratio of 0.3-1.4.

The aqueous fermentation medium preferably comprises 40-180 g/L, more preferably 50-150 g/L and most preferably 60-120 g/L of glucose equivalent of hydrolysed glucan.

According to a particularly preferred embodiment, the hydrolysed glucan in the aqueous fermentation medium is selected from hydrolysed amylose, hydrolysed amylopectin, hydrolysed cellulose, hydrolysed glycogen and combinations thereof. Most preferably the hydrolysed glucan comprises hydrolysed amylose and/or hydrolysed amylopectin. Both amylose and amylopectin are starch components.

According to a particularly preferred embodiment, the hydrolysed glucan is obtained by hydrolysis of wheat starch.

In a particularly preferred embodiment of the invention, the hydrolysed glucan employed in the present process is a partially hydrolysed starch, and during incubation the partially hydrolysed starch is further hydrolysed by added enzymes. Preferably, glucose constitutes not more than 70 wt. %, more preferably 10-60 wt. % of the partially hydrolysed starch.

Examples of enzymes that may be added to the fermentation medium to further hydrolyse the partially hydrolysed starch include glucoamylase, pullulanase, alpha-amylase, beta-amylase and combinations thereof. According to a particularly preferred embodiment, the added enzyme includes glucoamylase.

Examples of propionic acid producing micro-organism that may be employed in the present process include Acidipropionibacterium acidipropionici, Acidipropionibacterium freudenreichii, Acidipropionibacterium shermanii, Acidipropionibacterium thoenii, Acidipropionibacterium jensenii and combinations thereof. More preferably, the propionic acid micro-organism used is Acidipropionibacterium acidipropionici.

The incubation step is preferably carried out under anaerobic conditions.

The fermentation medium is preferably incubated at a temperature in the range of 20 to 45° C., more preferably of 25 to 42° C. and most preferably of 30 to 40° C.

The duration of the incubation step is preferably in the range of 22 to 120 hours, more preferably in the range of 24 to 72 hours.

In order to achieve a high yield of propionate, it is advantageous to continue the incubation step until virtually all hydrolysed glucan has been digested. Accordingly, in a preferred embodiment, incubation is continued until the fermentation medium contains no more than 10 g/L more preferably no more than 5 g/L, more preferably 0.1 to 3 g/L and most preferably 0.3 to 2 g/L of free glucose.

The incubation step is preferably continued until the propionate concentration in the fermentation medium has increased to at least 100 mmol/L, more preferably to at least 150 mmol/L and most preferably to 250-300 mmol/L.

The alkalising agent that is employed to control pH includes (i) an alkaline water-soluble alkalimetal salt and (ii) an alkaline water-soluble calcium salt. These two different alkalising agents may be added to the fermentation medium simultaneously or sequentially. Preferably the alkalimetal salt and the calcium salt are added simultaneously.

The alkaline water-soluble alkalimetal salt is preferably selected from sodium hydroxide, potassium hydroxide and combinations thereof.

The alkaline water-soluble calcium salt is preferably calcium hydroxide.

According to a particularly preferred embodiment of the present process, the alkalising agent is added to the fermentation medium to maintain pH within the range of 5.5 to 7.4 more preferably within the range of 5.8 to 7.2.

At the end of the incubation step the fermentate preferably comprises calcium cation and propionate in a molar ratio of 0.4 to 0.7, more preferably of 0.45 to 0.68.

Alkalimetal cation and propionate are preferably present in the fermentate at the end of the incubation step in a molar ratio of 0.4-1.2, more preferably of 0.5 to 1.0.

In accordance with a particularly preferred embodiment of the present invention, the present process contains the additional steps of:
removing undissolved solids from the fermentate by centrifugation or filtration to produce a low solids fermentate; and
drying the low solids fermentate.

The present process offers the advantage that it enables a surprisingly effective removal of undissolved solids, including biomass, thereby producing a low solids fermentate wherein most of the solute consists of propionate. Thus, a dried fermentate having a propionate content of more than 50% propionic acid equivalent can be obtained. Such a dried fermentate offers the additional advantage of having a low mean dynamic buffer capacity.

In the present process, undissolved solids may be removed by solid-liquid separation techniques known in the art, such as centrifugation, filtration, decanting and hydrocyclones. More preferably, the undissolved solids are removed by means of centrifugation or filtration. Most preferably, the undissolved solids are removed by means of disc stack centrifugation.

The inventors have observed that the undissolved solids that are removed from the fermentate include a significant amount of calcium salts. As a result, the molar ratio of calcium cation to propionate is substantially reduced by the removal of undissolved solids. The low solids fermentate that is obtained after removal of undissolved solids preferably comprises calcium cation and propionate in a molar ratio of 0.2 to 0.5, more preferably of 0.2 to 0.4.

The removal of undissolved solids does not strongly affect the molar ratio of alkalimetal cation to propionate. Preferably, alkalimetal cation and propionate are present in the low solids fermentate in a molar ratio of 0.3-1.4, more preferably of 0.4-1.2, most preferably of 0.5 to 1.0.

Following removal of the undissolved solids, the present process preferably comprises the additional step of concentrating the low solids fermentate to a dry solids content of at least 10 wt. %, more preferably of 12-40 wt. %, most preferably of 16-30 wt. %.

The low solids fermentate or the concentrated low solids fermentate is preferably dried to a moisture content of less than 15 wt. %, more preferably of less than 12 wt. % and most preferably of 1 to 10 wt. %.

The drying of the low solids fermentate or the concentrated low solids fermentate is preferably carried out by means of spray drying, drum drying, freeze drying or combination thereof. Most preferably the drying is carried out by means of spray drying.

According to a particularly preferred embodiment, the present process yields a dried fermentate as described herein before.

Another aspect of the invention relates to a method of preserving a food product or cosmetic product with the dried fermentate, wherein said method comprises incorporation of the dried fermentate of the present invention in a concentration of 0.1-5% by weight of the food product or cosmetic product, more preferably in a concentration of 0.2-2% by weight of the food product or cosmetic product.

The present method is particularly suited for preserving food products, especially bakery products and process meat products. Most preferably, the present method comprises addition of the dried fermentate to a bakery product.

Yet another aspect of the invention relates to a food product or a cosmetic product that is obtained by the present preservation method.

In a preferred embodiment, the product obtained by the preservation method is a food product, more preferably a food product selected from a bakery product and a processed meat product, most preferably a bakery product.

The food product of the present invention preferably has a hedonic liking score that is not significantly lower than that of a food product in which the dried fermentate has been replaced by a mixture of calcium propionate and starch.

The invention is further illustrated by the following non-limiting examples.

### EXAMPLES

### Example 1

Two dried propionate-containing fermentates were prepared starting from a fermentation medium that contained wheat starch syrup as the only fermentable carbohydrate source. The composition of the fermentation medium is shown in Table 1.

TABLE 1

| Compounds | Concentration [g/kg] |
|---|---|
| Glucose equivalent from wheat starch syrup | 70 |
| Yeast extract | 6.6 |
| Vitamins and minerals | 2.1 |
| Ammonia solution 28% w/v | 6.2 |
| Inoculum ¹ | 75.5 |
| Enzyme mixture ² | 1.1 |
| Water | Remainder |

¹ Contains Acidipropionibacterium acidipropionici
² Comprises glucoamylase, pullulanase, alpha-amylase and lysophospholipase

Water, magnesium sulphate, potassium sulphate, potassium dihydrogen phosphate, thiamine hydrochloride, biotin and calcium pantothenate were added to the fermenters and the trace element solution so obtained was in situ sterilized at 121° C. for 20 minutes.

Next, the fermenters were cooled down to ˜ 40° C. and an UHT-sterilised aqueous dilution of wheat starch syrup and yeast extract was added to the fermenters. The trace element solution was added aseptically to the fermenters and the bottles that contained the solution were subsequently rinsed with sterile demi water to ensure that all trace elements were transferred to the fermenter.

Prior to inoculation, the pH of the fermentation media was adjusted to pH 7.0 using ammonia solution. The initial glucose concentration was about 27 g/L.

Two different fermentation were carried out: Fermentation 1 was carried out according to the invention. Fermentation A was not according to the invention.

In Fermentation 1 ammonia solution and a mixture of Ca(OH)₂/NaOH (60:40 w/w) were used to control pH. The ammonia solution was added during the first 12 hours of fermentation. Once the ammonia solution was depleted, a switch was made towards the Ca(OH)₂/NaOH mixture. Fermentation A was conducted in the same manner, except that Ca(OH) 2 was used as sole base to control pH.

The fermentations were started by addition of the inoculum. After inoculation, the enzyme mixture was added aseptically to the fermenter and the bottles were subsequently rinsed with sterile demi water to ensure that all enzyme had been added.

The fermentations were carried out under sterile condition at pH 7.0, 35° C. and under constant agitation.

When the free glucose concentrations had reached 8 g/L (total glucose around 13 g/L), addition of base was stopped and the fermentation continued until pH 5.8 was reached. This procedure minimized carbonate formation, foaming during downstream processing and reduced the amount of solids in the final product.

The fermentates so obtained were concentrated, sterilised and spray dried, yielding Product 1 (from Fermentate 1) and Product A (from Fermentate A).

The compositions of the spray dried fermentates were analysed. The results are summarised in Table 2.

TABLE 2

Wt. %

| | Product 1 | Product A |
|---|---|---|
| Acetate | 7.2 | 7.4 |
| Lactate | 0.8 | 0.8 |
| Propionate | 31.5 | 30.0 |
| Succinate | 5.7 | 4.3 |
| Calcium | 11 | 18 |
| Sodium | 9.4 | 1.0 |
| Moisture | 2.0 | 2.4 |
| pH at 10%(w/v) dilution | 6.3 | 6.6 |

At least 95 wt. % of both spray dried fermentates had a particle size of not more than 300 μm. Carbohydrate analysis of Product 1 showed that it contained:
Glucose: 0.75 g/kg
Fructose: 0.1 g/kg
Isomaltose: 2.25 g/kg
Maltose: 0.35 g/kg

### Example 2

Example 1 was repeated to produce Fermentate 1. Next, biomass was removed from the fermentate by disc stack centrifugation, following which the clarified fermentate was concentrated, sterilised and spray dried.

The composition of the spray dried product (Product 2) The results are summarised in Table 3.

TABLE 3

| | Wt. % |
|---|---|
| Acetate | 14.7 |
| Lactate | 1.7 |
| Propionate | 50.4 |
| Succinate | 8.1 |
| Calcium | 8.3 |
| Sodium | 15 |
| Moisture | 1.7 |
| pH at 10%(w/v) dilution | 7.8 |

At least 95 wt. % of the spray dried fermentate had a particle size of not more than 300 μm.

### Example 3

The composition of the solids that had been removed in Example 2 via disc stack centrifugation was analysed. The results are presented in table 4.

TABLE 4

| | Wt. % |
|---|---|
| H₂O | 65.9 |
| CO₃²⁻/ HCO₃⁻ | 10.9 |
| Organic acids | 4.5 |
| Proteins | 4.4 |
| Glucose | 1.5 |
| Ca²⁺ | 2.7 |
| Remainder | 10.1 |

### Comparative Example A

Example 1 was repeated, except that this time, instead of the Ca(OH)₂/NaOH mixture, NaOH was used to control pH. It was found that in comparison to the Product 1 of Example 1, the product obtained (Product B) had a substantially lower propionate content and a much higher succinate content.

### Example 4

The fermentation experiments described in Example 1 were repeated. Product 3 was obtained by the same procedure as Product 1 of Example 1. Product C was obtained by the same procedure as Product 1 of Example A.

Products 3 and C were analysed. The results of these analyses are summarised in Table 5 together with the analytical results for Products 1 and A.

TABLE 5

| | Wt. % | | | |
|---|---|---|---|---|
| | Product 1 | Product A | Product 3 | Product C |
| Acetate | 7.2 | 7.4 | 7.6 | 7.4 |
| Lactate | 0.8 | 0.8 | 1.5 | 1.6 |
| Propionate | 31.5 | 30 | 26.9 | 27 |
| Succinate | 5.7 | 4.3 | 4.2 | 4.2 |
| Calcium | 11 | 18 | 10 | 18 |
| Sodium | 9.4 | 1 | 8.6 | 0.57 |
| Moisture | 2 | 2.4 | 2.9 | 2.8 |
| pH at 10%(w/v) dilution | 6.3 | 6.6 | 5.3 | 5.5 |

Each sample was dissolved in water to a concentration of 5% w/v. 10 untrained panellists evaluated each sample. Results are shown in Table 6. This table shows the mean scores for attribute Ranking Test using an anchored line scale labelled with “low” on the one end and “high” on the opposite end.

TABLE 6

| Solution | Bitter | Sweet | Astringent | Powdery |
|---|---|---|---|---|
| of | Intensity | Intensity | Intensity | Intensity |
| Product 1 | 32.9 | 32.25 | 29.3 | 30.2 |
| Product A | 45.6 | 26.8 | 28.4 | 30.5 |
| Product 3 | 17.3 | 31.8 | 22.0 | 28.9 |
| Product C | 49.8 | 23.7 | 34.2 | 34.1 |

### Example 5

The spray dried ferment samples of Example 4 were applied in a typical baked good, bread. The bread recipes are shown in Table 7 (ferments were added in amount providing 3.9 grams of propionic acid).

TABLE 7

| | Grams | | | |
|---|---|---|---|---|
| | Bread 1 | Bread A | Bread 2 | Bread B |
| Flour | 1000.00 | 1000.00 | 1000.00 | 1000.00 |
| Salt | 20.00 | 20.00 | 20.00 | 20.00 |
| Sugar | 80.00 | 80.00 | 80.00 | 80.00 |
| Anti-staling enzymes¹ | 5.00 | 5.00 | 5.00 | 5.00 |
| Dough conditioner² | 3.25 | 3.25 | 3.25 | 3.25 |
| Yeast - compressed | 120.00 | 120.00 | 120.00 | 120.00 |
| Soy oil | 20.00 | 20.00 | 20.00 | 20.00 |
| 60% lactic acid solution | 8.90 | 10.30 | 9.90 | 10.90 |
| Water | 566.00 | 566.00 | 566.00 | 566.00 |
| Product 1 | 12.38 | | | |
| Product A | | 13.00 | | |
| Product 3 | | | 14.50 | |
| Product C | | | | 14.45 |

¹Anti-staling enzymes can include ULTRAFRESH Premium 250, available from Corbion
²Dough conditioner can include PRISTINE Concentrate and PRISTINE Relaxer available from Corbion

The breads were prepared by the following process:
1. Mix dry ingredients
2. Add soy oil and lactic acid solution to dry ingredient mixture
3. Chill the water and add to mixing bowl
4. Add dry ingredients with soy oil to chilled water
5. Add yeast
6. Mix for 2 minutes on low speed
7. Mix 11-13 minutes on medium speed
8. Cover doughs with a towel and rest for 5 minutes
9. Cut into 6 portions, round, and allow to rest for another 5 minutes
10. Sheet, roll, and place into greased baking pan
11. Place in proofing box until dough height reaches proofing template
12. Bake at 420° F. (216° C.) for 23 minutes
13. Cool for 1 hour
14. Bag and seal

28 untrained panellists evaluated each bread sample for various aspects of liking. Results are shown in Table 8.

TABLE 8

| Bread | Overall | Aroma | Flavour | After-taste |
|---|---|---|---|---|
| 1 | 5.8 | 6.2 | 5.9 | 5.1 |
| A | 4.9 | 5.7 | 5.0 | 4.9 |
| 3 | 5.8 | 6.1 | 5.9 | 5.2 |
| C | 5.1 | 5.7 | 5.1 | 4.7 |

Notes regarding the testing:
1. The testing used a questionnaire with a 9-point scale where 9=Like extremely and 1=Dislike extremely and 5=Neither like nor dislike
2. Samples were tasted in pairs with Bread Samples 1 & A compared to each other and Bread Samples 2 & B compared to each other

### Example 6

A fermentate was produced in the same way as Fermentate 1 in Example 1. The fermentate was split into two portions. One portion was concentrated, sterilised and spray dried, yielding Product 4. The other portion was first centrifuged in a disc stack centrifuge, following which the supernatant was concentrated, sterilised and spray dried to produce Product 5.

Both products were analysed. The results are shown in Table 9.

TABLE 9

Wt. %

| | Product 4 | Product 5 |
|---|---|---|
| Acetate | 8.7 | 13.9 |
| Lactate | 0.8 | 1.2 |
| Propionate | 29.4 | 46.1 |
| Succinate | 6.2 | 10.0 |
| Calcium | 11.1 | 9.6 |
| Sodium | 9.3 | 14.2 |
| Moisture | 1.6 | 1.5 |
| pH at 10%(w/v) dilution | 6.4 | 5.5 |
| Mean dynamic buffer capacity | 29.7 | 23.8 |
| pH of maximum buffer capacity | Appr. 5.8 | 4.85 |

1. A dried fermentate having a moisture content of less than 15 wt. % and comprising, per kg of dry matter:
4.7-8.1 mol of propionate;
1.2-3.0 mol of calcium cation;
2.4-7.8 mol of alkali metal cation selected from sodium, potassium and combinations thereof; and
1-50 grams, glucose equivalent of hydrolysed glucan,
wherein the calcium cation and propionate are present in the dried fermentate in a molar ratio of 0.2 to 0.6.

2. The dried fermentate according to claim 1, wherein the fermentate comprises DNA originating from a propionic acid producing micro-organism.

3. The dried fermentate according to claim 1, wherein the fermentate comprises a bacterium belonging to the genus Acidipropionibacterium.

4. The dried fermentate according to claim 1, wherein the fermentate comprises a bacterium selected from Acidipropionibacterium acidipropionici, Acidipropionibacterium freudenreichii, Acidipropionibacterium shermanii, Acidipropionibacterium thoenii, Acidipropionibacterium jensenii, and combinations thereof.

5. The dried fermentate according to claim 1, wherein the fermentate comprises, per kg of dry matter, at least 3.4 mol.

6. The dried fermentate according to claim 1, wherein the fermentate further comprises, per kg of dry matter 1.5-3.0 mol of acetate.

7. The dried fermentate according to claim 1, wherein the fermentate further comprises, per kg of dry matter, 0.4-1.2 mol of succinate.

8. The dried fermentate according to claim 1, wherein the calcium cation and propionate are present in a molar ratio of 0.25 to 0.45.

9. The dried fermentate according to claim 1, wherein the calcium cation and alkali metal cation are present in a molar ratio of 0.25 to 0.62.

10. The dried fermentate according to claim 1, wherein the fermentate contains propionate in a concentration X, acetate in a concentration Y, succinate in a concentration Z, calcium cation in a concentration A, sodium cation in a concentration B, and potassium in a concentration C; wherein all said concentrations are expressed in mol/kg of dry matter; and wherein 0.8≤(X+Y+2Z)/(2A+B+C)≤1.2.

11. The dried fermentate according to claim 1, wherein dispersion of 100 grams of the dried fermentate in 1 litre of water yields an aqueous liquid having a pH in the range of 6 to 8.

12. The dried fermentate according to claim 1, wherein the dried fermentate has a mean dynamic buffer capacity of less than 27 mM/g.

13. The dried fermentate according to claim 1, wherein a solution that is obtained when the dried fermentate is dissolved in demineralised water of 20° C. to achieve a propionate concentration of 140 mmol/L has a bitterness score of less than 50 on a line scale from 0 to 100.

14. A process of producing a fermentate, the process comprising:
(a) providing an aqueous fermentation medium comprising 30-200 g/L glucose equivalent of hydrolysed glucan;
(b) inoculating the fermentation medium with a propionic acid producing micro-organism; and
(c) incubating the inoculated fermentation medium for at least 20 hours to produce a fermentate while adding an alkalising agent to maintain pH within the range of 5.0 to 7.5, wherein the alkalising agent comprises an alkaline water-soluble alkalimetal salt and an alkaline water-soluble calcium salt;
wherein the fermentate at the end of the incubation step comprises calcium cation and propionate in a molar ratio of 0.3 to 0.8 and alkalimetal cation and propionate in a molar ratio of 0.3-1.4.

15. The process according to claim 14, wherein the propionic acid producing micro-organism belongs to the genus Acidipropionibacterium.

16. The process according to claim 14, wherein the alkaline water-soluble alkalimetal salt is selected from sodium hydroxide, potassium hydroxide, and combinations thereof; and wherein the alkaline water-soluble calcium salt is calcium hydroxide.

17. The process according to claim 14, further comprising:
removing undissolved solids from the fermentate by centrifugation or filtration to produce a low solids fermentate; and
drying the low solids fermentate.

18. The process according to claim 14, wherein the process yields a fermentate having a moisture content of less than 15 wt. % and comprising per kg of dry matter:
4.7-8.1 mol of propionate;
1.2-3.0 mol of calcium cation;
2.4-7.8 mol of alkali metal cation selected from sodium, potassium and combinations thereof; and
1-50 grams glucose equivalent of hydrolysed glucan,
wherein the calcium cation and propionate are present in the fermentate in a molar ratio of 0.2 to 0.6.

19. A method of preserving a food product or cosmetic product, the method comprising incorporation of a dried fermentate according to claim 1 in a concentration of 0.1-5% by weight of the food product or cosmetic product.

20. A food product or a cosmetic product obtained by the method according to claim 19.
